(19) United States
(12) Patent Application Publication
(10) Pub. No.: US 2025/0143861 A1
(43) Pub. Date: May 8, 2025

(54) TORSIONAL COMPLIANCE

(71) Applicant: EDWARDS LIFESCIENCES CORPORATION, Irvine, CA (US)

(72) Inventor: Ido Halabi, Givatayim (IL)

(21) Appl. No.: 19/012,112

(22) Filed: Jan. 7, 2025

Related U.S. Application Data
(63) Continuation of application No. PCT/US2023/070974, filed on Jul. 25, 2023.
(60) Provisional application No. 63/370,101, filed on Aug. 1, 2022.

(51) Int. Cl. A61F 2/02

(52) U.S. Cl. CPC A61F 2/02; A61F 2220/0008

(57) ABSTRACT

Devices, systems, and/or methods can provide compliance characteristics to fluid vessels. For example, an implant device can be advanced to a target site in a fluid vessel, such as an aorta. The implant device can include a first anchoring element, a second anchoring element, and/or a torsion element coupled between the first anchoring element and the second anchoring element. The first and/or second anchoring element of the implant device can be deployed at the target site. In one example, the implant device can be used to bias an aorta toward a twisted state for mimicking a healthy aorta and thereby improving blood flow therethrough.

### RELATED APPLICATION(S)

This application is a continuation of International Application No. PCT/US2023/070974, filed Jul. 25, 2023, which claims the benefit of U.S. Provisional Patent Application No. 63/370,101, filed on Aug. 1, 2022, the complete disclosures of which are hereby incorporated by reference in their entireties.

### BACKGROUND

### Field

The present disclosure generally relates to the field of medical implant devices.

### Description of Related Art

Insufficient or reduced compliance in certain blood vessels, including arteries such as the aorta, can result in reduced perfusion, cardiac output, and other health complications. Restoring compliance and/or otherwise controlling flow in such blood vessels can improve patient outcomes.

### SUMMARY

Described herein are devices, methods, and/or systems that facilitate the restoration of compliance characteristics to undesirably stiff blood vessels. Devices associated with the various examples of the present disclosure can include one or more anchoring elements to anchor to a fluid vessel and one or more torsion elements coupled to the one or more anchoring elements and configured to twist and/or untwist the fluid vessel to provide a change in volume of the fluid vessel over the cardiac cycle. Such change in volume can allow the blood vessel to mimic compliance of a healthy blood vessel and/or otherwise promote blood flow during, for example, a phase of the cardiac cycle.

For purposes of summarizing the disclosure, certain aspects, advantages, and/or features are described. It is to be understood that not necessarily all such advantages may be achieved in accordance with any particular example. Thus, the disclosed examples can be carried out in a manner that achieves or optimizes one advantage or group of advantages as taught herein without necessarily achieving other advantages as may be taught or suggested herein.

### BRIEF DESCRIPTION OF THE DRAWINGS

Various examples are depicted in the accompanying drawings for illustrative purposes. In addition, various features of different disclosed examples can be combined to form additional examples, which are part of this disclosure. Throughout the drawings, reference numbers may be reused to indicate correspondence between reference elements.

FIG. 1A illustrates an example representation of a heart and associated vasculature having various features relevant to one or more examples of the present disclosure.

FIG. 1B-1 illustrates an example healthy aorta.

FIG. 1B-2 illustrates an example unhealthy aorta.

FIGS. 2A-1 and 2B-1 provide side and cross-sectional views, respectively, of a compliant blood vessel radially contracting/recoiling during the diastolic phase of the cardiac cycle.

FIGS. 2A-2 and 2B-2 provide side and cross-sectional views, respectively, of a compliant blood vessel experiencing expansion during the systolic phase of the cardiac cycle.

FIGS. 3-1 and 3-2 illustrate cross-sectional views of a blood vessel that is relatively stiff.

FIG. 4 is a graph illustrating blood pressure over time in an example healthy patient.

FIG. 5 is a graph illustrating blood pressure over time in an example patient having reduced aortic compliance.

FIGS. 6A, 6B, and 6C provide perspective, side, and front views, respectively, of an example implant device that can be configured to apply torsional force to a fluid vessel.

FIG. 7-1 illustrates an example implant device that includes a tube disposed within anchoring features.

FIG. 7-2 illustrates an example implant device that includes a tube disposed around anchoring features.

FIGS. 8A-1 and 8A-2 provide side and cross-sectional views, respectively, of a blood vessel and implant device in a substantially straight/untwisted state.

FIGS. 8B-1 and 8B-2 provide side and cross-sectional views, respectively, of a blood vessel and implant device in an at least partially twisted state.

FIGS. 9-1, 9-2, and 9-3 illustrate a flow diagram for a process for implanting an implant device in accordance with one or more examples.

FIGS. 10-1, 10-2, and 10-3 provide images of the implant device and certain anatomy corresponding to operations of the process of FIGS. 9-1, 9-2, and 9-3 according to one or more examples.

FIG. 11 illustrates an example implant device in a fully deployed state within an aorta.

FIG. 12 illustrates an example implant device in a fully deployed state around an aorta.

### DETAILED DESCRIPTION

The headings provided herein are for convenience and do not necessarily affect the scope or meaning of the subject matter.

Although certain examples are disclosed below, the subject matter extends beyond the specifically disclosed examples to other alternative examples and/or uses and to modifications and equivalents thereof. Thus, the scope of the claims that can arise here from is not limited by any of the examples described below. In any method or process disclosed herein, the acts or operations of the method or process can be performed in any suitable sequence and are not necessarily limited to any particular disclosed sequence. Various operations can be described as multiple discrete operations in turn, in a manner that can be helpful in understanding certain examples; however, the order of description should not be construed to imply that these operations are order dependent. Additionally, the structures, systems, and/or devices described herein can be embodied as integrated components or as separate components. For purposes of comparing various examples, certain aspects of these examples are described. Not necessarily all such aspects or advantages are achieved by any particular example. Thus, for example, various examples can be carried out in a manner that achieves or optimizes one advantage or group of advantages as taught herein without necessarily achieving other aspects or advantages as can also be taught or suggested herein.

Certain reference numbers are re-used across different figures of the figure set of the present disclosure as a matter of convenience for devices, components, systems, features, and/or modules having features that can be similar in one or more respects. However, with respect to any of the examples disclosed herein, re-use of common reference numbers in the drawings does not necessarily indicate that such features, devices, components, or modules are identical or similar. Rather, one having ordinary skill in the art can be informed by context with respect to the degree to which usage of common reference numbers can imply similarity between referenced subject matter. Use of a particular reference number in the context of the description of a particular figure can relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another.

Where an alphanumeric reference identifier is used that comprises a numeric portion and an alphabetic portion (e.g., ‘10a,’ ‘10’ is the numeric portion and ‘a’ is the alphabetic portion), references in the written description to the numeric portion (e.g., ‘10’) can refer to any feature identified in the figures using such numeric portion (e.g., ‘10a,’ ‘10b,’ ‘10c,’ etc.), even where such features are identified with reference identifiers that concatenate the numeric portion thereof with one or more alphabetic characters (e.g., ‘a,’ ‘b,’ ‘c,’ etc.). That is, a reference in the present disclosure to a feature ‘10’ can be refer to either an identified feature ‘10a’ in a particular figure of the present disclosure or to an identifier ‘10’ or ‘10b’ in the same figure or another figure, as an example.

Certain standard anatomical terms of location are used herein to refer to the anatomy of animals, and namely humans, with respect to various examples. Although certain spatially relative terms, such as “outer,” “inner,” “upper,” “lower,” “below,” “above,” “vertical,” “horizontal,” “top,” “bottom,” and similar terms, are used herein to describe a spatial relationship of one device/element or anatomical structure to another device/element or anatomical structure, these terms are used herein for ease of description to describe the positional relationship between element(s)/structures(s), as illustrated in the drawings. Spatially relative terms are generally intended to encompass different orientations of the element(s)/structures(s), in use or operation, in addition to the orientations depicted in the drawings. For example, an element/structure described as “above” another element/structure can represent a position that is below or beside such other element/structure with respect to alternate orientations of the subject patient or element/structure, and vice-versa. Spatially relative terms, including those listed above, can be relative to a respective illustrated orientation of a referenced figure.

### Vascular Anatomy and Compliance

Certain examples are disclosed herein in the context of vascular implant devices, and in particular, compliance-enhancement implant devices implanted in the aorta. However, although certain principles disclosed herein can be particularly applicable to the anatomy of the aorta, the compliance-enhancement implant devices in accordance with the present disclosure can be implanted in, or configured for implantation in, any suitable or desirable blood vessels or other anatomy, such as the inferior vena cava, etc.

The anatomy of the heart and vascular system is described below to assist in the understanding of certain concepts disclosed herein. In humans and other vertebrate animals, the heart generally comprises a muscular organ having four pumping chambers, wherein the flow thereof is at least partially controlled by various heart valves, namely, the aortic, mitral (or bicuspid), tricuspid, and pulmonary valves. The valves can be configured to open and close in response to a pressure gradient present during various stages of the cardiac cycle (e.g., relaxation and contraction) to at least partially control the flow of blood to a respective region of the heart and/or to blood vessels (e.g., ventricles, pulmonary artery, aorta, etc.). The contraction of the various heart muscles can be prompted by signals generated by the electrical system of the heart.

Figures IA illustrates an example representation of a heart 100 and associated vasculature having various features relevant to one or more examples of the present disclosure. The heart 100 includes four chambers, namely the left atrium 102, the left ventricle 104, the right ventricle 106, and the right atrium 108. In terms of blood flow, blood generally flows from the right ventricle 106 into the pulmonary artery 110 via the pulmonary valve 112, which separates the right ventricle 106 from the pulmonary artery 110 and is configured to open during systole so that blood can be pumped toward the lungs and close during diastole to prevent blood from leaking back into the heart from the pulmonary artery 110. The pulmonary artery 110 carries deoxygenated blood from the right side of the heart 100 to the lungs. The pulmonary artery 110 includes a pulmonary trunk and left and right pulmonary arteries that branch off of the pulmonary trunk, as shown.

The tricuspid valve 114 separates the right atrium 108 from the right ventricle 106. The tricuspid valve 114 generally has three cusps/leaflets and can generally close during ventricular contraction (i.e., systole) and open during ventricular expansion (i.e., diastole). The mitral valve 116 generally has two cusps/leaflets and separates the left atrium 102 from the left ventricle 104. The mitral valve 116 is configured to open during diastole so that blood in the left atrium 102 can flow into the left ventricle 104, and, when functioning properly, closes during systole to prevent blood from leaking back into the left atrium 102. The aortic valve 118 separates the left ventricle 104 from the aorta 120. The aortic valve 118 is configured to open during systole to allow blood leaving the left ventricle 104 to enter the aorta 120, and close during diastole to prevent blood from leaking back into the left ventricle 104.

The heart valves can generally comprise a relatively dense fibrous ring, referred to herein as the annulus, as well as a plurality of leaflets or cusps attached to the annulus. Generally, the size of the leaflets or cusps can be such that when the heart contracts the resulting increased blood pressure produced within the corresponding heart chamber forces the leaflets at least partially open to allow flow from the heart chamber. As the pressure in the heart chamber subsides, the pressure in the subsequent chamber or blood vessel can become dominant and press back against the leaflets. As a result, the leaflets/cusps come in apposition to each other, thereby closing the flow passage. Disfunction of a heart valve and/or associated leaflets (e.g., pulmonary valve disfunction) can result in valve leakage and/or other health complications.

The atrioventricular (mitral and tricuspid) heart valves generally are coupled to a collection of chordae tendineae and papillary muscles (not shown for visual clarity) for securing the leaflets of the respective valves to promote and/or facilitate proper coaptation of the valve leaflets and prevent prolapse thereof. The papillary muscles, for example, can generally comprise finger-like projections from the ventricle wall. The valve leaflets are connected to the papillary muscles by the chordae tendineae. A wall of muscle, referred to as the septum, separates the left 102 and right 108 atria and the left 104 and right 106 ventricles.

The vasculature of the human body, which can be referred to as the circulatory system, cardiovascular system, or vascular system, contains a complex network of blood vessels with various structures and functions and includes various veins (venous system) and arteries (arterial system). Generally, arteries, such as the aorta, carry blood away from the heart, whereas veins, such as the inferior and superior venae cavae, carry blood back to the heart.

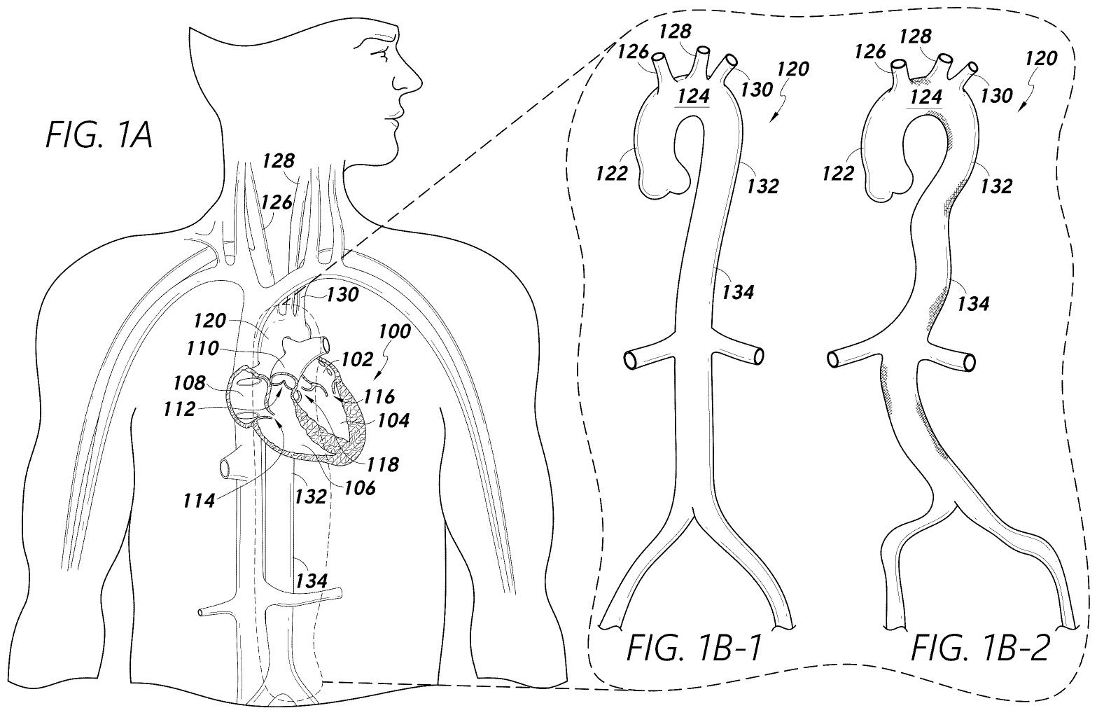

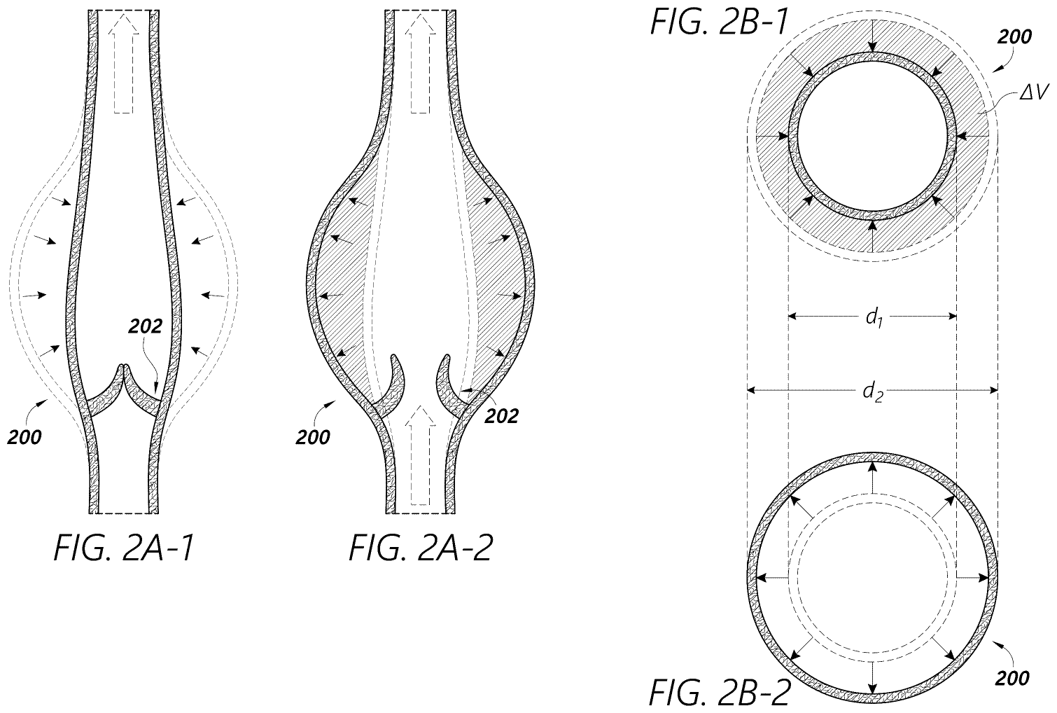

FIGS. 1B-1 and 1B-2 show detailed views of example healthy and aged/stiff aortas 120, respectively. The aorta 120 is a compliant arterial blood vessel that buffers and conducts pulsatile left ventricular output and contributes the largest component of total compliance of the arterial tree. The aorta 120 includes the ascending aorta 122, which begins at the opening of the aortic valve 118 in the left ventricle 104 of the heart 100. The ascending aorta 122 and pulmonary trunk 110 twist around each other, causing the aorta 120 to start out posterior to the pulmonary trunk 110, but end by twisting to its right and anterior side. Among the various segments of the aorta 120, the ascending aorta 122 is relatively more frequently affected by aneurysms and dissections, often requiring open heart surgery to be repaired. The transition from ascending aorta 122 to aortic arch 124 is at the pericardial reflection on the aorta. At the root of the ascending aorta 122, the lumen has three small pockets between the cusps of the aortic valve 118 and the wall of the aorta 120, which are called the aortic sinuses or the sinuses of Valsalva. The left aortic sinus contains the origin of the left coronary artery and the right aortic sinus likewise gives rise to the right coronary artery. Together, these two arteries supply the heart with blood.

As mentioned above, the aorta 120 is coupled to the heart 100 via the aortic valve 118, which leads into the ascending aorta 122 and gives rise to the innominate artery 126, the left common carotid artery 128, and the left subclavian artery 130 along the aortic arch 124 before continuing as the descending thoracic aorta 132 and further the abdominal aorta 134. References herein to the aorta can be understood to refer to the ascending aorta 122 (also referred to as the “ascending thoracic aorta”), aortic arch 124, descending or thoracic aorta 132 (also referred to as the “descending thoracic aorta”), abdominal aorta 134, or other arterial blood vessel or portion thereof.

Arteries, such as the aorta 120, can utilize blood vessel compliance (e.g., arterial compliance) to store and release energy through the stretching of blood vessel walls. The term “compliance” can be used herein according to its broad and ordinary meaning, and can refer to the ability of an arterial blood vessel or prosthetic implant device to distend, expand, stretch, or otherwise deform in a manner as to increase in volume in response to increasing transmural pressure, and/or the tendency of a blood vessel (e.g., artery) or prosthetic implant device, or portion thereof, to recoil toward its original dimensions as transmural pressure decreases.

Arterial compliance facilitates perfusion of organs in the body with oxygenated blood from the heart. Generally, a healthy aorta and other major arteries in the body are at least partially elastic and compliant, such that they can act as a reservoir for blood, filling up with blood when the heart contracts during systole and continuing to generate pressure and push blood to the organs of the body during diastole. In older individuals and patients suffering from heart failure and/or atherosclerosis, compliance of the aorta and other arteries can be diminished to some degree or lost. Such reduction in compliance can reduce the supply of blood to the organs of the body due to the decrease in blood flow during diastole. Among the risks associated with insufficient arterial compliance, a significant risk presented in such patients is a reduction in blood supply to the heart muscle itself. For example, during systole, generally little or no blood can flow in the coronary arteries and into the heart muscle due to the contraction of the heart which holds the heart at relatively high pressures. During diastole, the heart muscle generally relaxes and allows flow into the coronary arteries. Therefore, perfusion of the heart muscle relies on diastolic flow, and therefore on aortic/arterial compliance.

A healthy aorta, as shown in FIG. 1B-1, runs along a generally straight path, whereas an aged and/or stiffened aorta, as shown in FIG. 1B-2, can run along a more tortuous, curved path. That is, the aorta tends to change in shape as a function of age, resulting in higher degrees of curvature or tortuosity, as developed gradually over time. Such change in shape of the blood vessel can be associated with the vasculature of the subject becoming less elastic. As such conditions develop, arterial blood pressure (e.g., left-ventricular afterload) can become more pulsatile, which can have deleterious effects, such as the thickening of the left ventricle (LV) muscle, and insufficient perfusion of the heart. Insufficient perfusion of the heart muscle can lead to and/or be associated with heart failure. Heart failure is a clinical syndrome characterized by certain symptoms, including breathlessness, ankle swelling, fatigue, and others. Heart failure may be accompanied by certain signs, including elevated jugular venous pressure, pulmonary crackles, and peripheral edema, for example, which may be caused by structural and/or functional cardiac abnormality. Such conditions can result in reduced cardiac output and/or elevated intra-cardiac pressures at rest or during stress.

FIGS. 2A-1 and 2B-1 provide side and cross-sectional views, respectively, of a compliant blood vessel 200, such as an artery (e.g., aorta), radially contracting/recoiling during the diastolic phase of the cardiac cycle. FIGS. 2A-2 and 2B-2 provide side and cross-sectional views, respectively, of the compliant blood vessel 200 experiencing expansion during the systolic phase of the cardiac cycle. As understood by those having ordinary skill in the art, the systolic phase of the cardiac cycle is associated with the pumping phase of the left ventricle, while the diastolic phase of the cardiac cycle is associated with the filling phase of the left ventricle. As identified in FIG. 2B-1, with proper arterial compliance, a change in volume ΔV will generally occur in an artery between high- and low-pressure phases of the cardiac cycle. With respect to the aorta, as shown in FIGS. 2A and 2B, as blood is pumped into the aorta 200 through the aortic valve 202, the pressure in the aorta increases and the diameter of at least a portion of the aorta expands. A first portion of the blood entering the aorta 200 during systole may pass through the aorta during the systolic phase, while a second portion (e.g., approximately half of the total blood volume) may be stored in the expanded volume ΔV caused by compliant stretching of the blood vessel, thereby storing energy for contributing to perfusion during the diastolic phase. A compliant aorta may generally stretch with each heartbeat, such that the diameter of at least a portion of the aorta expands.

The tendency of the arteries to stretch in response to pressure as a result of arterial compliance can have a significant effect on perfusion and/or blood pressure in some patients. For example, arteries with relatively higher compliance can be conditioned to more easily deform than lower-compliance arteries under the same pressure conditions. Compliance (C) can be calculated using the following equation, where ΔV is the change in volume (e.g., in mL) of the blood vessel, and ΔP is the pulse pressure from systole to diastole (e.g., in mmHg):

$$C = \frac{Δ V}{Δ P} \qquad ( 1 )$$

Aortic stiffness and reduced compliance can lead to elevated systolic blood pressure, which can in turn lead to elevated intracardiac pressures, increased afterload, and/or other complications that can exacerbate heart failure. Aortic stiffness further can lead to reduced diastolic flow, which can lead to reduced coronary perfusion, decreased cardiac supply, and/or other complications that can likewise exacerbate heart failure.

Arterial compliance restoration devices, methods, and concepts disclosed herein may be generally described in the context of the ascending aorta. However, such devices, methods and/or concepts can be applicable in connection with any other artery or blood vessel.

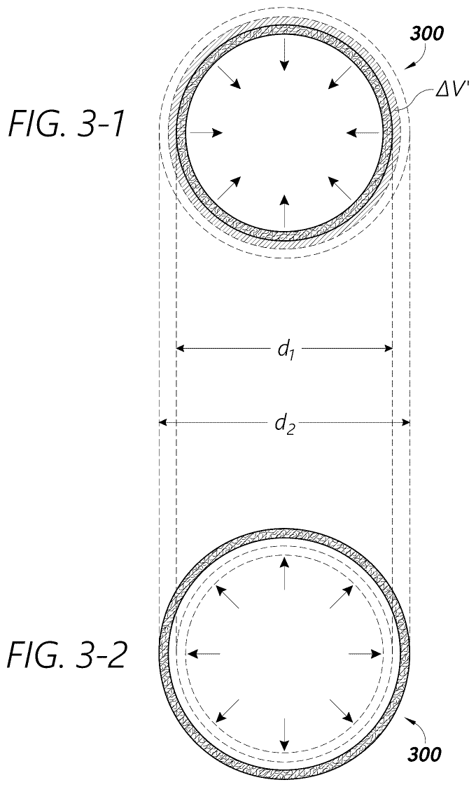

FIGS. 3-1 and 3-2 show a cross-sectional profile of a blood vessel 300 that is relatively stiff, such as the blood vessel shown in FIG. 1B-2, wherein the compliance of the vessel portion 300 is diminished relative to the healthy aorta as shown in FIG. 1B-1. Due to the stiffness of the blood vessel wall, the blood vessel 300 can expand a relatively limited amount between diastole (shown in FIG. 3-1) and systole (shown in FIG. 3-2). That is, during systole, the increased fluid pressure within the blood vessel 300 can result in a relatively small and/or negligible expansion of the diameter of the blood vessel 300, as shown with respect to the difference between the contracted diameter d₁ and the expanded diameter d₂. Due to the limited expansion of the blood vessel 300, the change in volume ΔV′ in the blood vessel between phases of the cardiac cycle can likewise be limited, and therefore relatively little energy is stored in the blood vessel wall and returned to the blood circulation during low-pressure conditions, resulting in more pulsatile blood flow compared to healthy, compliant aortic tissue.

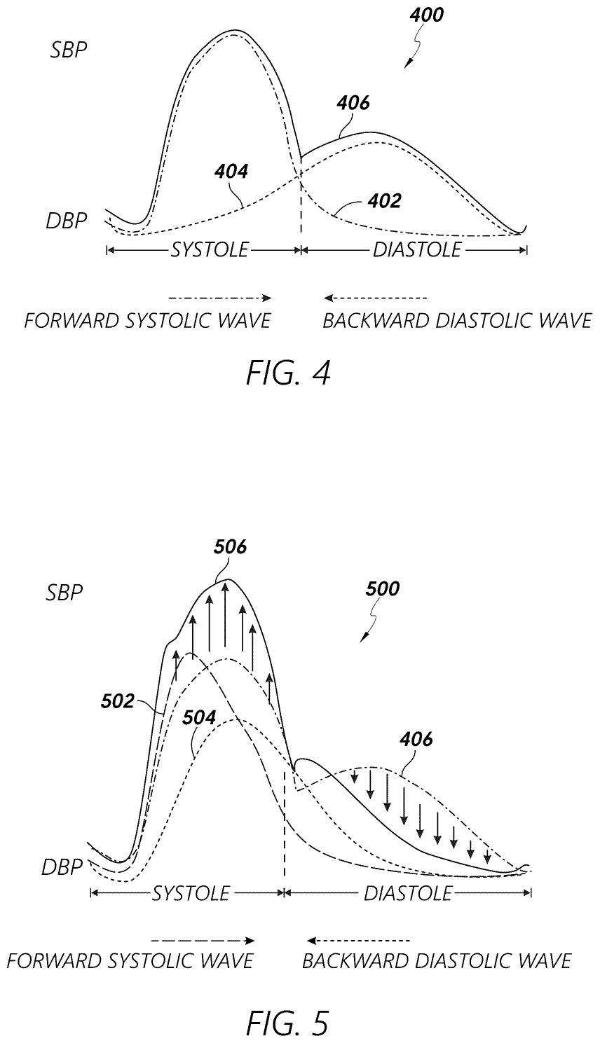

FIG. 4 is a graph 400 illustrating blood pressure over time in an example patient with a healthy, compliant aorta, wherein arterial blood pressure is represented as a combination of a forward systolic pressure wave 402 and a backward diastolic pressure wave 404. The combination of the systolic wave 402 and the diastolic wave 404 are represented by the waveform 406.

FIG. 5 is a graph 500 illustrating blood pressure over time in an example patient having reduced aortic compliance. The graph 500 shows, for reference purposes, the example combined wave 406 shown in FIG. 4. When low compliance is exhibited, less energy can be stored in the aorta compared to a healthy patient. Therefore, the systolic waveform 502 can demonstrate increased pressure during the systolic phase relative to a patient having normal compliance, while the diastolic waveform 504 can demonstrate reduced pressure during the diastolic phase relative to a patient having normal compliance. Therefore, the resulting combined waveform 506 can represent an increase in the systolic peak and a drop in the diastolic pressure, which can cause various health complications. For example, the change in waveform can impact the workload on the left ventricle and can adversely affect coronary profusion.

In view of the health complications that can be associated with reduced arterial compliance, as described above, it can be desirable in certain patients and/or under certain conditions, to at least partially alter compliance properties of the aorta or other artery or blood vessel, or otherwise alter/control flow therein, in order to improve cardiac and/or other organ health.

### Compliance-Enhancing Devices

The present disclosure relates to systems, devices, and methods for at least partially restoring and/or increasing compliance to a blood vessel, such as the aorta or other arterial (or venous) blood vessel(s), to provide improved perfusion of the heart muscle and/or other organ(s) of the body. Examples of the present disclosure can include devices configured to be implanted within or around a fluid vessel and configured to apply a torsional force to the fluid vessel to thereby twist and untwist the fluid vessel with a change in force of a fluid flowing through the vessel (e.g., luminal pressure). Such twisting-untwisting movement can cause the volume within the fluid vessel to change, allowing the blood vessel to mimic compliance of a healthy vessel and/or otherwise promote flow during phases of the cardiac cycle. For example, the devices can apply a torsional force to cause the fluid vessel to move towards a twisted state (having a smaller cross-sectional area) during a lower-pressure period and allow the vessel to move towards an untwisted state (having a larger cross-sectional area) during a higher-pressure period. Thus, the fluid vessel can expand and store energy during higher-pressure periods of the cardiac cycle (e.g., during the systolic phase/period) and contract/compress during lower-pressure periods (e.g., during the diastolic phase/period) to return the stored energy to the circulation and increase flow through the vessel. In examples, the devices discussed herein can reduce pulsatile left ventricle afterload.

Methods and/or structures disclosed herein for treating a patient also encompass analogous methods and structures performed on or placed on a simulated patient, which is useful, for example, for training, for demonstration, for procedure and/or device development, and the like. The simulated patient can be physical, virtual, or a combination of physical and virtual. A simulation can include a simulation of all or a portion of a patient, for example, an entire body, a portion of a body (e.g., thorax), a system (e.g., cardiovascular system), an organ (e.g., heart), or any combination thereof. Physical elements can be natural, including human or animal cadavers, or portions thereof, synthetic, or any combination of natural and synthetic. Virtual elements can be entirely in silica or overlaid on one or more of the physical components. Virtual elements can be presented on any combination of screens, headsets, holographically, projected, loudspeakers, headphones, pressure transducers, temperature transducers, or using any combination of suitable technologies.

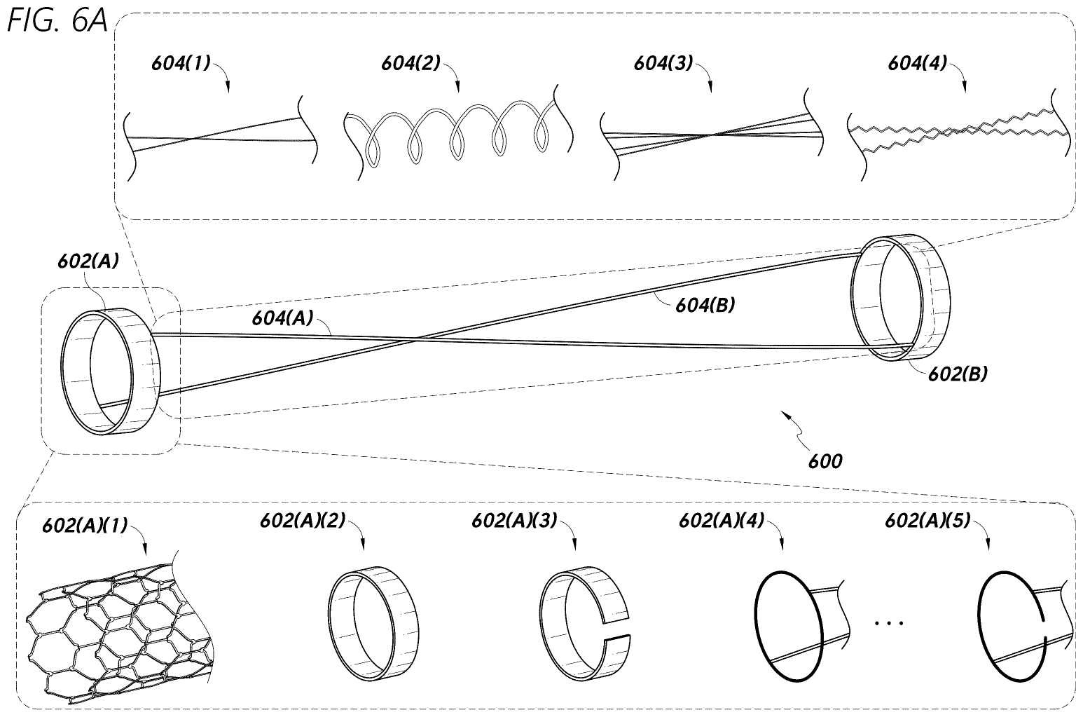

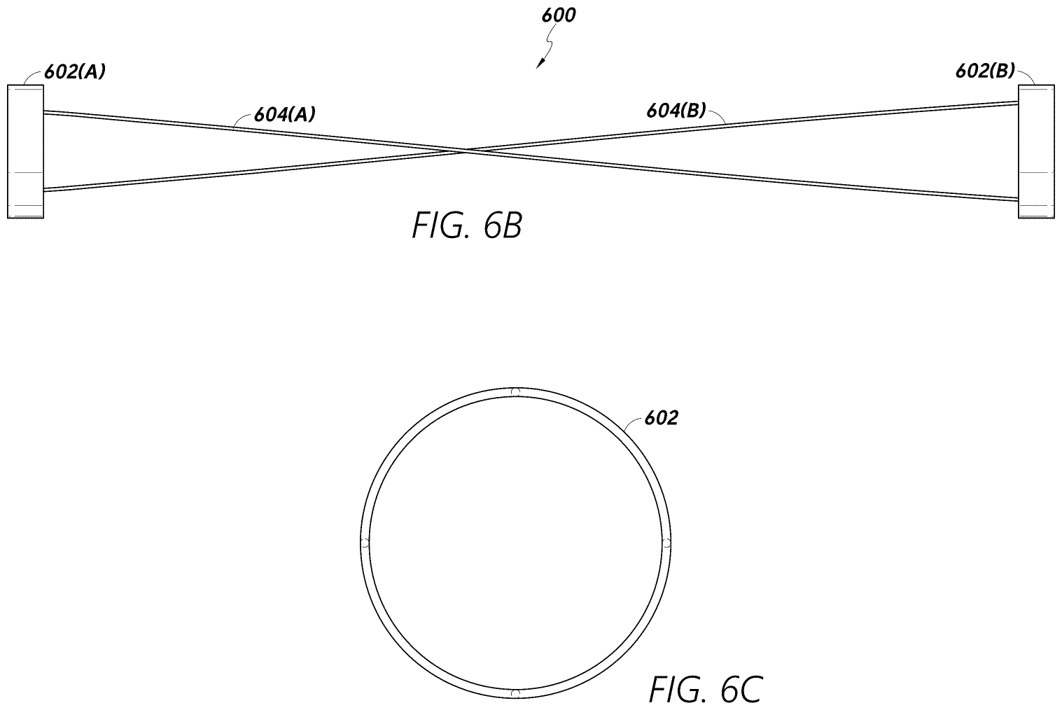

FIGS. 6A-6C provide perspective, side, and front views, respectively, of an example device/system 600 that can be configured to apply torsional/torsion force to a fluid vessel (also referred to as “the implant device 600”). The device 600 can include one or more anchoring features/elements/structures 602 (also referred to as “the one or more anchors 602” or “the anchoring/securing means 602”) to secure the implant device 600 to anatomy of a patient and one or more torsion elements/structures 604 (also referred to as “the torsion means 604”) coupled/attached to the anchoring features 602. The one or more anchoring features 602 (sometimes referred to as “the anchoring features 602” for convenience), the one or more torsion elements 604 (sometimes referred to as “the torsion element 604” for convenience), and/or other features of the device 600 can couple to a fluid vessel (not shown), such as an artery, vein, etc. The device 600 can facilitate twisting/untwisting of the fluid vessel based on luminal pressure within the fluid vessel, thereby changing volume of the fluid vessel and providing additional compliance to the fluid vessel as the luminal pressure changes.

The torsion element 604 can be configured to bias the device 600 and/or the associated fluid vessel to a particular state. For example, the torsion element 604 can be configured to bias the device 600 and/or the fluid vessel towards a twisted state/biased position (or untwisted state, in some cases), such that device 600 applies a torsional force/torque to the anchoring features 602 and/or the fluid vessel when the device 600 is untwisted/rotated away from a biased position (e.g., when the anchoring features 602 are rotated in opposite directions relative to each other). To illustrate, the device 600 can be configured to cause/allow the fluid vessel to be twisted for lower luminal pressures within the fluid vessel and cause/allow the fluid vessel to untwist/straighten for higher luminal pressures. In some examples, torsion refers to the act of twisting or the state of being twisted, which can occur due to opposite torques applied/experienced at separate ends/positions of an item/anatomy.

In one example where the device 600 is attached to the aorta, the device 600 can be coupled to the aorta in a manner that causes the aorta to twist (e.g., aortic torsion) during diastole when the pressure is lower within the aorta. Here, the device 600 biases the fluid vessel to a twisted/torsion state. During systole, the blood pressure in the aorta can increase to cause the walls of the aorta to expand and overcome the biasing force of the device 600, thereby untwisting/straightening the device 600 and aorta. As the blood pressure decreases, the biasing force of the device 600 overcomes the force of the walls of the aorta and causes the aorta to twist again. Thus, the volume within the aorta can increase during at least a portion of a first cardiac phase (e.g., systolic phase) (due to the untwisting) and decrease during at least a portion of a second cardiac phase (e.g., a diastolic phase) (due to the twisting), thereby providing compliance characteristics for the aorta. In this illustration, the torsional force applied by the device 600 can be configured/designed to, during diastole, overcome the resistive torsional force of the aorta to remain straight and/or configured/designed to, during systole, allow the luminal pressure within the aorta and torsional force of the aorta (to straighten) to untwist the aorta.

Although various examples are discussed in the context of biasing towards a twisted state, the device 600 can alternatively be biased towards an untwisted state, such that the device 600 is configured to apply a torsional force to untwist a fluid vessel. Further, although various examples illustrate the implant device 600 with two anchoring features 602 and two torsion elements 604, the device 600 can be implemented with any number of anchoring features and/or torsion elements. In some examples, one or more of the anchoring features 602 and one or more of the torsion elements 604 are integrated/implemented together.

The device 600 can be shaped to conform to the shape/form of the associated anatomy in which the device 600 is implanted. In various examples discussed herein, such as that illustrated in FIGS. 6A-6C, the anchoring features 602 and/or the torsion element 604 form a cylindrical frame/structure. Here, when implanted at a substantially straight portion of the aorta, the device 600 can have a substantially straight cylindrical form. However, the device 600 can have a bent shape (e.g., along the longitudinal axis of the device 600) and/or can be at least partially flexible to adapt to the attached anatomy. For instance, if the device 600 is implanted in the aortic arch, the device 600 can bend to conform to the curvature of the aortic arch. In some examples, the device 600 can include additional features/structure/frame elements, such as a tube as discussed in further detail below.

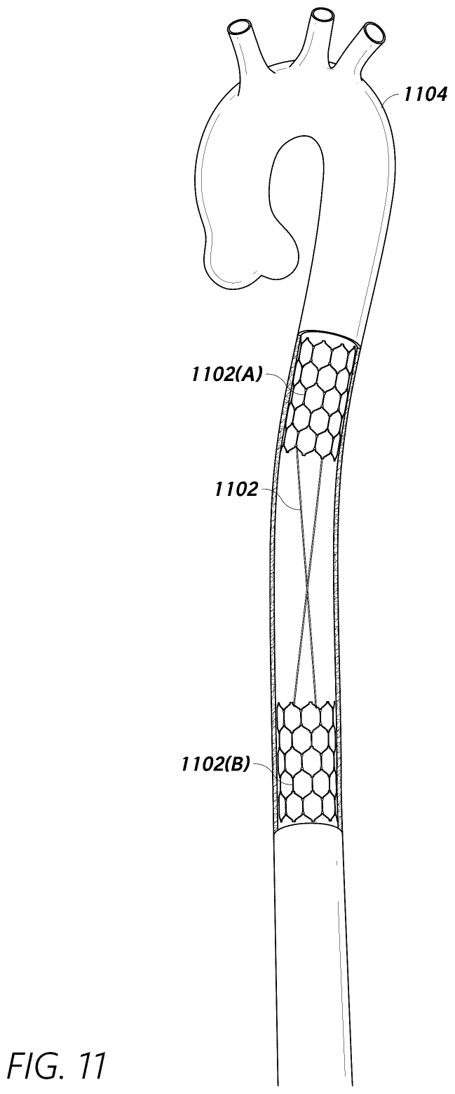

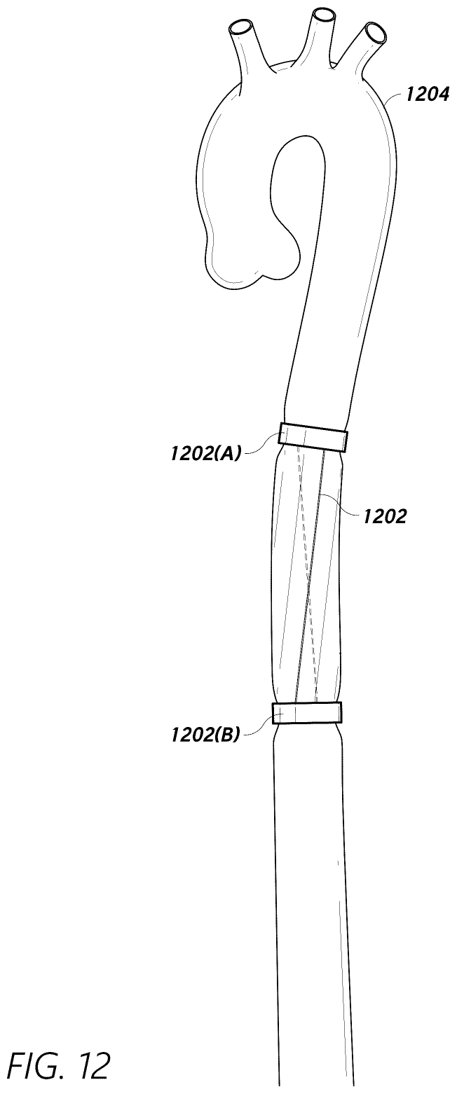

The anchoring features 602 can be implemented in a variety of manners to anchor/secure/attach the implant device 600 to the target tissue. The anchoring features 602 can secure the device 600 to an internal surface/portion of a fluid vessel, an outer/external surface/portion of the fluid vessel, or a combination thereof, as shown in the examples of FIGS. 11 and 12. In examples, the anchoring features 602 can be implemented as elements that are self-expandable, balloon/device expandable, self-contractible, device contractible, crimp-able, or otherwise configured to expand, contract, or deform to couple to the anatomy. In some instances, the anchoring features 602 are attached to tissue in a self-expanding/contracting manner. Alternatively, or additionally, the anchoring features 602 can be manipulated by a device/physician to attach to the tissue. In examples, the device 600 can be configured to be compressed (e.g., radially compressed) and transported within a delivery catheter/sheath or other tubular delivery system, such as in the case of a minimally invasive procedure through a percutaneous opening or natural orifice. As such, the device 600 can be a percutaneously-placeable implant. Alternatively, the device 600 can be implanted through another type of procedure, such as an open surgery.

In some instances, the anchoring features 602 include barbs, patches, pins, coils, screws, tabs, hooks, wires, or other tissue anchor means configured to embed in and/or hold to the respective wall portion of the fluid vessel. For example, the anchoring features 602 can have wires or barbs having free ends that project radially outward to puncture the tissue of the native blood vessel and secure the device 600 in place. In some implementations, such wires/barbs have shape-memory that predisposes such structure to deflect radially outwardly once deployed from a capsule/sheath to facilitate anchoring of the device 600 to the native blood vessel.

As shown in FIG. 6A, in examples the anchoring feature 602(A) (which is representative of any of the anchoring features 602) is implemented as a stent/frame structure 602(A)(1). The frame 602(A)(1) can have a structure comprising a plurality of struts forming an array of cells, which can have any suitable or desirable shape (e.g., oval/ellipse, diamond/rhombus, hexagonal diamond/polygon, etc.). The cells can be arranged in any number of columns in the circumferential direction and rows in the axial, or lengthwise, direction. The frame 602(A)(1) can be formed using any suitable process, such as by stamping or machining the frame structure from a sheet or tube of metal. The frame 602(A)(1) can be made of any at least partially rigid material, such as metal, plastic, etc. For example, the frame 602(A)(1) can comprise stainless steel or nitinol. Where nitinol or other shape-memory metal or material is implemented, the frame 602(A)(1) can be self-expanding. For instance, an expandable stent-type frame can be configured to expand radially from a compressed delivery configuration to the expanded state shown at 602(A)(1). In some implementations, the array of struts is formed from a sheet of metal, which is rolled into a cylinder to form the tubular/cylindrical form of the frame 602(A)(1) configured for placement within a blood vessel. In some implementations, a balloon catheter is used for delivery and/or to expand the frame 602(A)(1) for securing in the wall of an artery or other blood vessel or body cavity.

In some cases of implementing the stent/frame 602(A)(1) as the anchoring feature 602(A) (and/or in other types of anchoring features), the outside or inside of the frame 602(A)(1) is covered with a fabric, polymer cover, or other material. The covering/cover can promote tissue ingrowth within the inner diameter of the native blood vessel. The cover can be disposed on an outer surface or area of the frame 602(A)(1), and/or can be disposed/applied to the inner diameter of the frame 602(A)(1) on an inside thereof. In some implementations, the cover comprises a cloth or polymer sleeve which may be at least partially elastic, or alternatively nonelastic. The cover can be applied over or within the frame 602(A)(1) in any suitable or desirable manner. For example, the cover can be applied using an electrical or mechanical spinning (e.g., rotary jet spinning, electrospinning, or similar) application process or other deposition process.

Further, in examples, the anchoring feature 602(A) is implemented as a ring/band, which can be a continuous band 602(A)(2) or a non-continuous band 602(A)(3) having a gap/break/space in the ring. In some instances, the band 602(A)(2)/602(A)(3) (also referred to as “the band 602(A)” for convenience) is surgically attached to a fluid vessel, such as an outer/external surface/portion of the vessel. However, the band 602(A) can be implanted in other cases, such as through a minimally invasive procedure/transcatheter approach/delivery. The band 602(A) can be made of an at least partially rigid material, such as a metal, plastic, or another material. In some cases, a non-continuous band 602(A)(3) is implanted on the vessel and the gap/spacing between the ends of the band 602(A)(3) is filled in or the ends of the band are otherwise attached/coupled together to form the continuous band 602(A)(2). For example, during a procedure, the non-continuous band 602(A)(3) can be manipulated/enlarged to position the band 602(A)(3) around a blood vessel. The band 602(A)(3) can then be compressed/deformed such that the open ends are brought into contact with each other. The ends can be fastened/adhered/soldered or otherwise attached to each other to form the continuous band 602(A)(2), which can prevent the band 602(A) from slipping on the blood vessel. In examples, the ends of the band 602(A)(3) include attachment elements/means, such as clips, fasteners, etc. to attach/couple the ends to each other. Alternatively, the band 602(A) can be inserted into an internal portion of the blood vessel and expanded to contact/embed into the internal wall of the blood vessel. As such, the band 602(A) can be a compressible or expandable band to assist in securing the device 600 to the tissue.

Moreover, the anchoring feature 602(A) can be implemented as a wire/flexible rod, which can be a continuous wire 602(A)(4) or a non-continuous wire 602(A)(5) having a gap/break/space. In some instances, the wire 602(A)(4)/602(A)(5) (also referred to as “the wire 602(A)” for convenience) is compressible, such that the wire 602(A) is configured to be delivered to a target site in an at least partially compressed/delivery state and deployed/expanded to an expanded state, as shown in FIG. 6A. For example, a delivery catheter can be used to advance the device 600 to a delivery site in a compressed configuration. The wires 602(A) can be anchored to an internal surface or outer surface of the blood vessel. In the case of external surface anchoring, a puncture hole can be created with another device or the wire 602(A)(5) and the wire 602(A)(5) can be navigated through the tissue wall to the external surface by rotating the device 600/wire 602(A)(5). However, the wire 602(A) can be surgically implanted or implanted in other manners. The wire 602(A) can be made of an at least partially rigid material, such as a metal, plastic, or another material. In some cases, a non-continuous wire 602(A)(5) is implanted on the vessel and the ends of the wire 602(A) are attached/coupled together to form the continuous wire 602(A)(4). Although the figures illustrate particular types of anchoring features, any type of anchoring feature can be implemented.

The torsion element 604 can be implemented in a variety of manners to bias the device 600 to a particular configuration/orientation. For example, the torsion element/means 604 can include a torsion spring, which can include a torsion bar(s)/wire(s)/rod(s), helical torsion spring(s), torsion fiber(s), etc. To illustrate, the torsion element/means 604 can be implemented as two bars/wires/rods (as shown at 604(1) in FIG. 6A and elsewhere), a helical shape/form (as shown at 604(2)), more than two bars/wires/rods (as shown at 604(3)), non-straight bars/wires/rods (as shown at 604(4)), etc. These examples illustrate some of many implementations. Each torsion element/bar/wire can take a relatively straight path from one anchoring feature 602(A) to another anchoring feature 602(B) (as shown in various example figures herein) or another path.

The torsion element 604 can include an at least partially flexible item/material that is configured to store energy (e.g., apply torsional force) when the torsion element 604 is twisted (relative to its longitudinal axis, for example). For instance, the torsion element 604 can exert/apply torsional force/torque in an opposite relation/direction relative to the direction in which the torsion element 604 is twisted. To illustrate, if a first end of the device 600 is twisted/rotated in a first rotational direction relative to a second end of the device 600, the torsion element 604 can exert a torque/rotational force to cause the first end to rotate back in an opposite rotational direction. In some examples, twisting or untwisting movement is referred to as a torquing motion. To illustrate, a first torquing motion can refer to the device 600 twisting (e.g., a first end rotating in a first direction), while a second torquing motion can refer to the device 600 untwisting (e.g., the first end rotating in a second, opposite direction), or vice versa. The torsion element 604 can be coupled/attached to the anchoring features 602 and/or integral with the anchoring features 602. Although illustrated as including less than a single coil/turn in FIG. 6A (and other figures), the torsion element 604 can include any number of coils/turns.

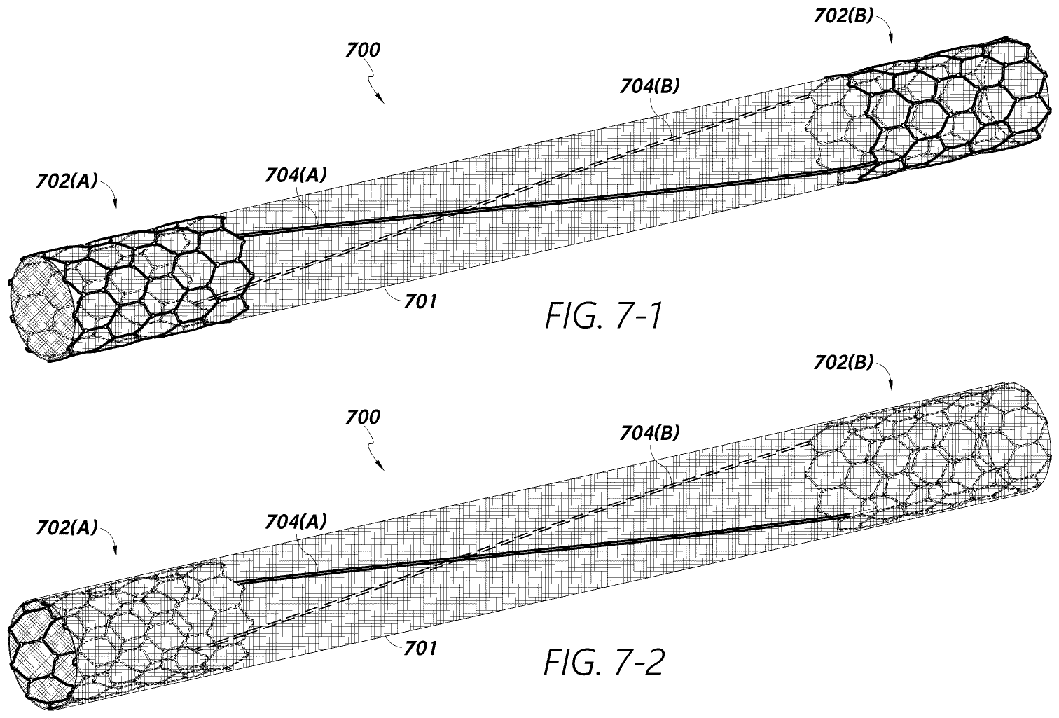

FIGS. 7-1 and 7-2 illustrate an example implant device 700 that includes a tube/sleeve/tubular structure 701 to provide additional structure and/or compliance characteristics to the device 700. FIG. 7-1 illustrates an example where the tube 701 is disposed within one or more anchoring features 702, while FIG. 7-2 illustrate an example where the tube 701 is disposed around the one or more anchoring features 702. The tube 701 can generally be cylindrical in shape/form. As such, the tube 701 can be referred to as a cylindrical tube 701 or a cylindrical elongate member. However, the tube 701 can take other shapes/forms.

The tube 701 can be constructed of a compliant/elastic material, such as an elastomeric polymer or other material configured to twist and untwist and/or radially expand/stretch and contract/recoil. For instance, the tube 701 (also referred to as “the compliant or elastic tube 701”) can include a fabric/cloth material or balloon structure including a thermoplastic polyurethane (TPU), nylon, etc. In some examples, the tube 701 comprises a woven structure, such as a woven memory metal braided structure, or the like. In some examples, the tube 701 comprises biological tissue. The tube 701 can assist in providing a change in volume of the associated fluid vessel in which the device 700 is implanted, such as by twisting and untwisting with the device 600 as the fluid vessel twists and untwists.

The tube 701 and can be attached to or integral with the one or more anchoring features 702 and/or one or more torsion elements 704. The one or more anchoring features 702 can be similar to or the same as the one or more anchoring features 602 discussed in reference to FIGS. 6A-6C, while the one or more torsion elements 704 can be similar to or the same as the one or more torsion elements 604 also discussed in reference to FIGS. 6A-6C. In the example of FIG. 7-1, the tube 701 is disposed/positioned within the anchoring features 702 and/or the torsion elements 704. Meanwhile, the example of FIG. 7-2 illustrates the tube 701 disposed/positioned outside of the anchoring features 702 and/or the torsion elements 704. The torsion element 604 (e.g., spring) can be coiled around or within the tube 701 any number of times.

In some cases, the tube 701 can be compressed to implement a collapsed state for delivery of the device 700 to a target site using a delivery device, such as a catheter, sheath, etc. To illustrate, the device 700 can be radially compressed and/or twisted to decrease an outer diameter of the device 700 to fit within a delivery system to facilitate transcatheter access. Once released from the delivery system, the device 700 can expand to the form illustrated in FIGS. 7-1 and 7-2.

Although various examples are discussed in the context of implanting the device 700 within or around a fluid vessel, the device 700 can alternatively be implemented as part of the fluid vessel. For instance, a portion of the fluid vessel can be removed/cut and the open ends of the vessel can be coupled/attached to the ends of the device 700, such that the device 700 functions as part of the fluid vessel (e.g., a graft). The tube 701 can provide a path for a fluid/blood to flow through the fluid vessel (e.g., connecting the severed ends of the fluid vessel). The tube 701 can be configured to twist and untwist, based on luminal pressure, to provide compliance for the fluid vessel.

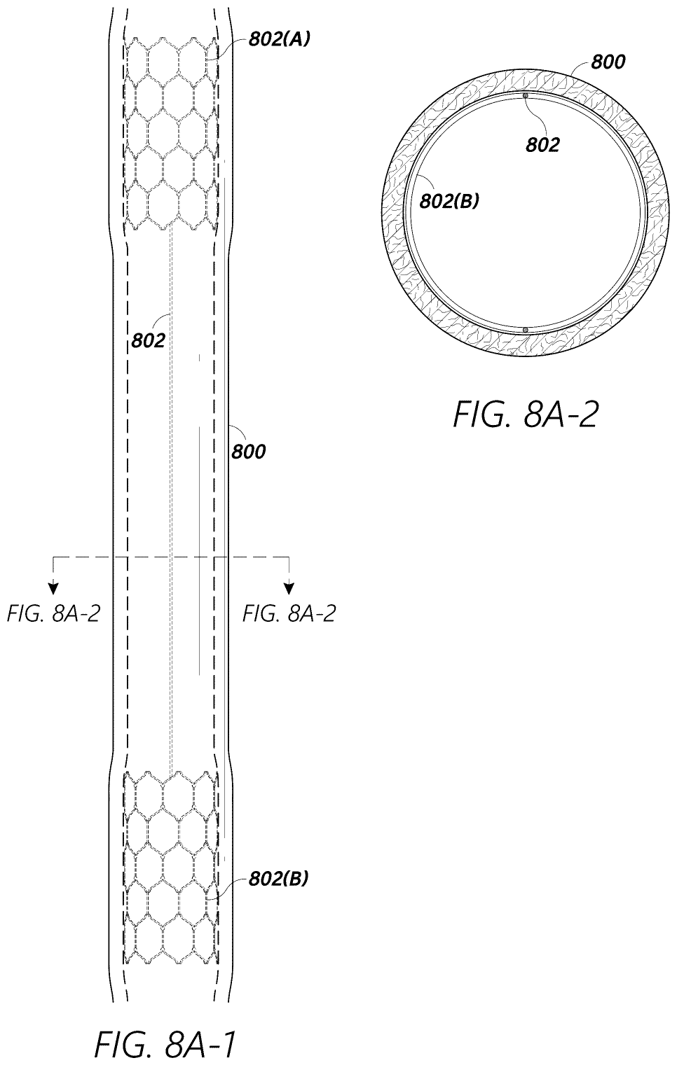

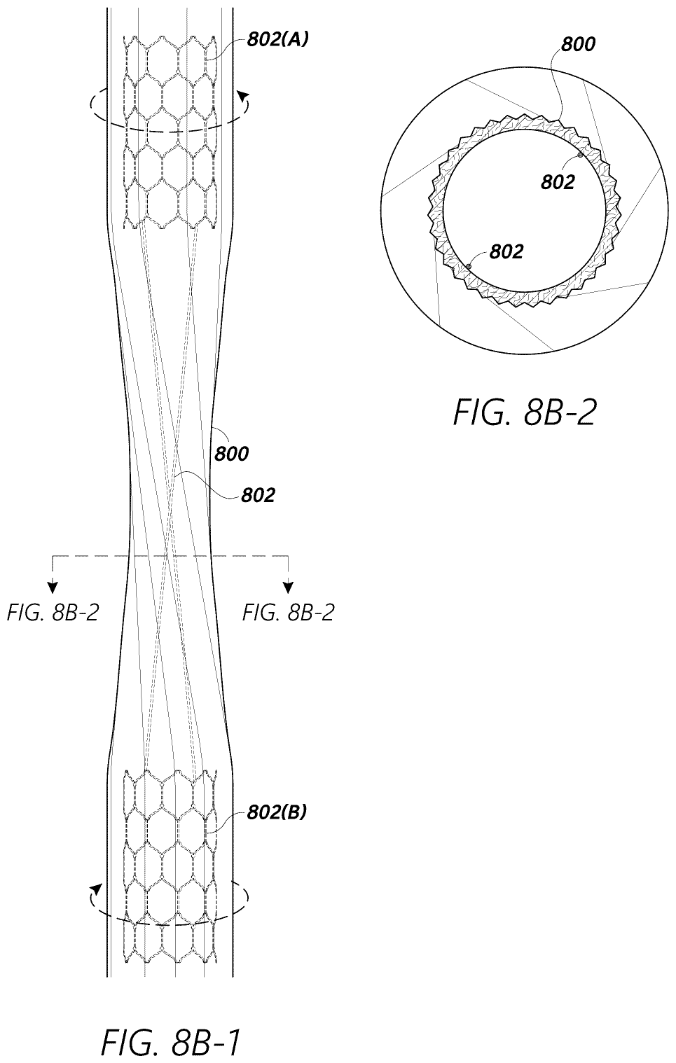

FIGS. 8A-1, 8A-2, 8B-1, and 8B-2 provide various views of an example blood vessel 800 to illustrate a change in cross-sectional area and volume of the blood vessel 800 that occurs as an implant device 802 facilitates twisting and twisting of the blood vessel 800 during various stages of a cardiac cycle. FIGS. 8A-1 and 8A-2 provide side and cross-sectional views, respectively, of the blood vessel 800, such as an artery (e.g., aorta), in a substantially straight/untwisted state, which can occur during a systolic phase of the cardiac cycle when blood pressure (e.g., aortic pressure) is relatively high. FIGS. 8A-2 and 8B-2 provide side and cross-sectional views, respectively, of the blood vessel 800 in an at least partially twisted/torsion state, which can occur during a diastolic phase of the cardiac cycle when blood pressure (e.g., aortic pressure) is relatively low. The implant device 802 shown in the figures can be similar to or the same as any of the implant devices discussed herein.

The implant device 802 (and/or any of the other implant devices discussed herein) can be configured to exert torque to one or more portions of the blood vessel 800 an in attempt to maintain the blood vessel 800 in a twisted state, such as that shown in FIG. 8B-1. That is, the implant device 802 is configured to bias the blood vessel 800/device 802 to a twisted state. An amount/degree of twisting of the blood vessel 800/device 802 can be based on an amount of luminal/blood pressure within the blood vessel 800, an amount of torsional force that the device 802 is configured to apply/exert, and/or an amount of force of the blood vessel 800 to resist twisting (which can be based on the characteristics of the blood vessel 800). An amount/degree of twisting can refer to an amount of rotation of a first end of the implant device 802 (or first location/portion of the blood vessel 800) relative to a second end of the implant device 802 (or second location/portion of the blood vessel 800) or another reference point. Although discussed in the context of biasing towards a twisted state, the implant device 802 can exert an opposite force/torque in to attempt to maintain the blood vessel 800 in a straight state (e.g., to bias the blood vessel 800 to an untwisted state).

As shown in FIGS. 8A-2 and 8B-2, the cross-sectional area of the interior portion of the blood vessel 800 (e.g., the blood flow path) changes as the vessel 800 twists and untwists. For instances, when the blood/luminal pressure is relatively low in the blood vessel 800 (e.g., during a diastolic phase), the torsional/biasing force of the device 802 can overcome the blood pressure (and/or the resistive force of the blood vessel 800) and cause the vessel 800 to twist. In the twisted state, the cross-sectional area of the blood vessel 800, and ultimately the volume within the blood vessel 800, is relatively small (e.g., smaller than in the straight state), as shown in FIG. 8B-2. This can be due to the smaller diameter/circumference of the cross-section. In examples, when the blood vessel 800 is twisted, the wall of the vessel 800 can be somewhat deformed, distorted, warped, folded, or otherwise changed in shape/form.

As the blood/luminal pressure increases within the blood vessel 800 (e.g., during a systolic phase) and overcomes the biasing force of the device 802, the blood vessel 800 straightens/untwists, as shown in FIG. 8A-1. In the straight state, the cross-sectional area of the blood vessel 800, and ultimately the volume within the blood vessel 800, is relatively large (e.g., larger than in the twisted state), as shown in FIG. 8A-2. In the straight state, energy is stored within the device 802 and/or the blood vessel 800 in the form of a torsional force. As this stored energy is released, the blood vessel 800 returns to the twisted state. This twisting process assists in pumping blood through the vessel 800. That is, the stored energy can be returned to the blood circulation by decreasing the volume in the blood channel. This twisting and untwisting can occur with the natural cardiac cycle to change in volume of the blood vessel 800, thereby improving the compliance of the blood vessel 800.

The implant device 802 (and/or any of the other implant devices discussed herein) can be configured to twist a blood vessel by any amount (e.g., bias the blood vessel to any amount of twisting). For instance, in a fully twisted state (also referred to as a biased/default state) (as shown in FIG. 8B-1), a first end 802(A) of the implant device 802 can be rotated around a longitudinal axis of the device 802 any number of degrees, such as 5 degrees, 10 degrees, 15 degrees, 20 degrees, 30 degrees, 45 degrees, 60 degrees, 90 degrees, 120 degrees, 180 degrees, 360 degrees, and so on, relative to a reference point and/or second end 802(B) of the device 802. In the twisted/biased state, the first end 802(A) (e.g., first anchoring structure) can be positioned/oriented at a biased position relative to the second end 802(B) (e.g., second anchoring structure) or another reference point. As such, a first portion of the blood vessel 800 (where the first end 802(A) of the device 600 is attached) can be configured to rotate relative to a second portion of the blood vessel 800 (where the second end 802(B) of the device 600 is attached) by any amount. In some examples, the implant device 802 is configured to twist the blood vessel 800 by more than a full rotation/twist (e.g., by more than 360 degrees).

An amount of rotation/twist (or untwist) that an implant device is configured to implement (e.g., for different forces) can be designed/based on one or more characteristics, such as an amount of desired blood volume change, a length of the implant device, a current amount of blood flow/volume for a blood vessel, etc. The device 802 can be designed to satisfy certain criteria, such as beginning to twist when the blood vessel 800 experiences less than a threshold luminal pressure, beginning to untwist when the blood vessel experiences more than a threshold luminal pressure, etc.

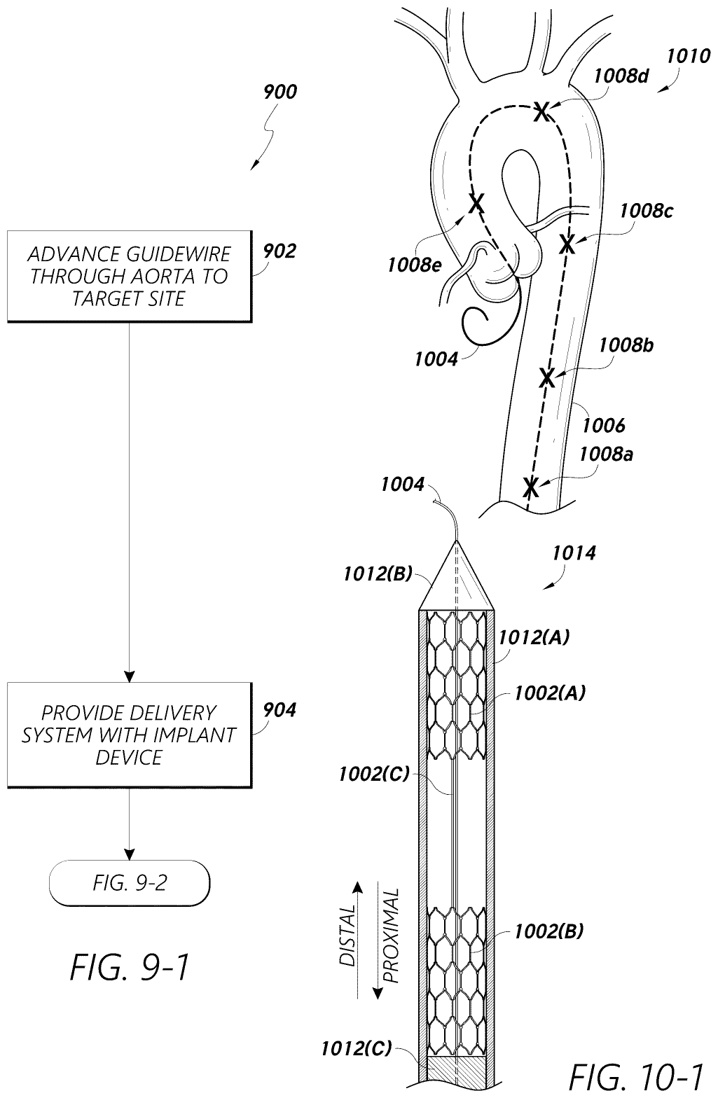

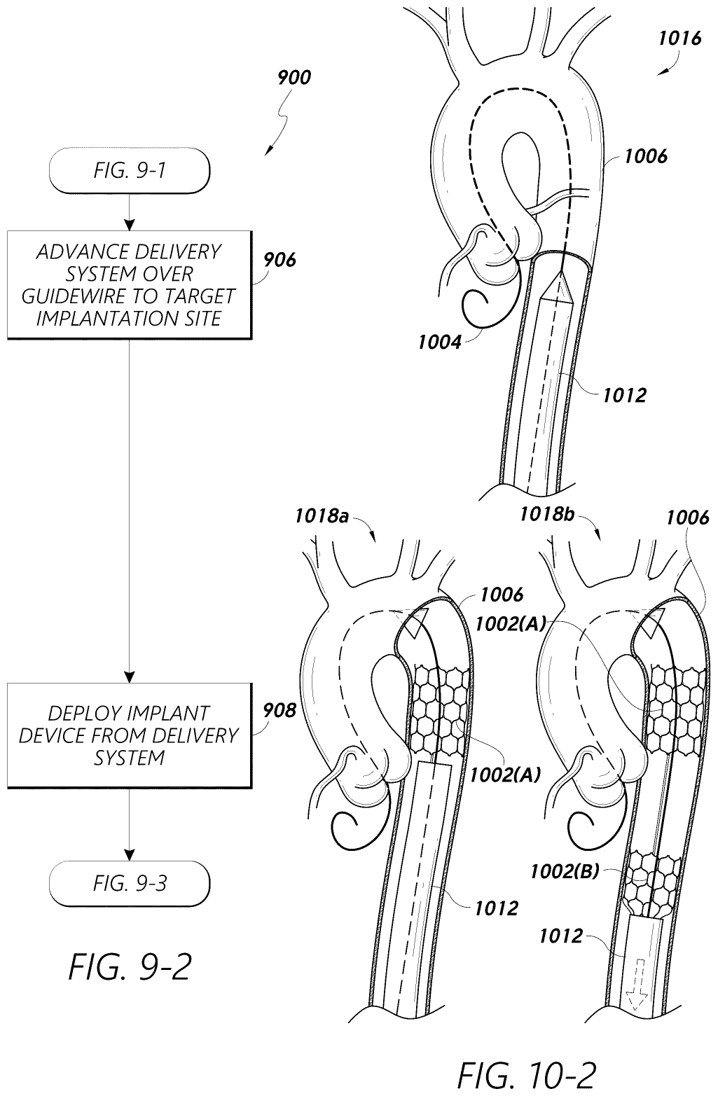

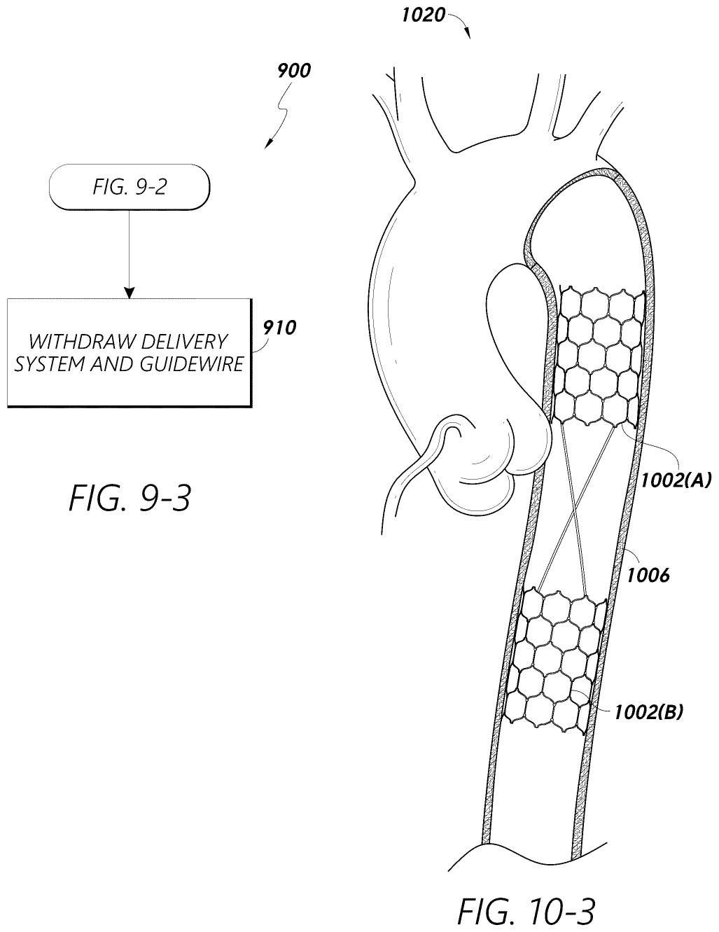

FIGS. 9-1, 9-2, and 9-3 illustrate a flow diagram for a process 900 for implanting an implant device 1002 (which can be similar to or the same as any of the implant devices discussed herein) in accordance with one or more examples. FIGS. 10-1, 10-2, and 10-3 provide images of the implant device 1002 and certain anatomy corresponding to operations of the process 900 of FIGS. 9-1, 9-2, and 9-3 according to one or more examples. The process 900 utilizes a transcatheter procedure for implantation/deployment of compliance-enhancement implant devices in accordance with aspects of the present disclosure. However, implant devices disclosed herein can be implanted using other types of minimally-invasive and/or surgical procedures.

At block 902, the process 900 involves advancing a guidewire 1004 through at least a portion of the aorta 1006 of the patient to reach a target implantation site 1008. For example, image 1010 shows an example implantation site 1008a in the abdominal aorta 1006, example implantation sites 1008b, 1008c in the descending thoracic aorta 1006, an example implantation site 1008d in the aortic arch 1006, and an example implantation site 1008e in the ascending aorta 1006, such as in the area of the aortic valve. The guidewire 1004 can be advanced through the aortic valve, or to any point along the path of the aorta 1006. Access to the aorta 1006 can be made through any suitable vessel puncture providing access to the arterial system. For example, access can be made via the femoral artery or other arterial blood vessel. In some implementations, access is made to the inferior vena cava via the femoral vein or other access, wherein a guidewire and/or other instrumentation can be crossed over into the abdominal aorta 1006 in an area where the inferior vena cava and abdominal aorta 1006 are adjacent to one another by puncturing through the venous wall and the arterial wall and advancing through such puncture openings.

Although the process 900 and certain other examples are described herein in the context of implantation within the aorta 1006, compliance-enhancement devices of the present disclosure can be implanted in other arterial or venous blood vessels, such as the inferior vena cava. Further, although the process 900 and accompanying illustrations are presented with respect to the implantation of a single compliance-enhancement implant device 1002, the process 900 can involve implanting multiple compliance-enhancement implant devices in various positions within the aorta 1006 and/or other blood vessel(s).

At block 904, the process 900 involves providing a delivery system 1012 having a compliance-enhancement implant device 1002 disposed in a distal portion thereof. Image 1014 of FIG. 10-1 shows a cut-away view of an example implementation of the delivery system 1012 in accordance with one or more examples. The delivery system 1012 can comprise one or more catheters or sheaths used to advance and/or implant the compliance-enhancement implant device 1002, which can be disposed at least partially within the delivery system 1012 during portions of the process 900. The implant device 1002 can be positioned within the delivery system 1012 with a first end thereof (e.g., inflow end at an anchoring feature 1002(A)) disposed distally and a second end (e.g., outflow end at an anchoring feature 1002(B)) disposed proximally with respect to the illustrated orientation of the delivery system 1012.

In some examples, the delivery system 1012 comprises an outer catheter/shaft/sheath 1012(A), which can be used to transport the compliance-enhancement implant device 1002 to the target implantation site. That is, the compliance-enhancement implant device 1002 can be advanced to the target implantation site at least partially within a lumen of the outer shaft 1012(A), such that the compliance-enhancement implant device 1002 is held and/or secured at least partially within a distal portion of the outer shaft 1012(A) in a radially compressed configuration.

In some examples, the delivery system 1012 comprises a tapered nosecone feature 1012(C), which can facilitate advancement of the distal end of the delivery system 1012 through the tortuous anatomy of the patient and/or an outer delivery sheath or other conduit/path. The nosecone 1012(C) can be a separate component from the outer shaft 1012(A) or can be integrated with the outer shaft 1012(A). In some examples, the nosecone 1012(C) is adjacent to and/or integrated with a distal end of the outer shaft 1012(A). In some examples, the nosecone 1012(C) is distally tapered into a generally conical shape and can comprise and/or be formed of multiple flap-type forms that can be urged/spread apart when the compliance-enhancement implant device 1002 and/or any portions thereof, interior shafts, or devices, are advanced distally therethrough.

The delivery system 1012 can further be configured to have the guidewire 1004 disposed at least partially within the delivery system 1012 and/or coupled thereto in a manner to allow the delivery system 1012 to follow a path defined by the guidewire 1004. In some implementations, the guidewire 1004 can pass through an interior of the implant device 1002 and/or through a lumen of a pusher device or tube of the delivery system 1012. In examples, the delivery system 1012 can include/implement a delivery capsule to hold the device 1002.

The compliance-enhancement implant device 1002 can have any of the features of the examples described in detail herein, including the first anchoring feature 1002(A), second anchoring feature 1002(B), and/or torsion element 1002(C), which may or may not include a sleeve/tube couple thereto (not shown for ease of illustration). In this example, the anchoring features 1002(A) and 1002(B) are implemented as stents/wire frames; however, the anchoring features 1002(A) and 1002(B) can be implemented in other manners, as discussed herein. The implant device 1002 can be disposed within the shaft/sheath 1012(A) in a radially compressed/collapsed configuration, wherein the anchoring feature 1002(A) and/or anchoring feature 1002(B) are crimped to assume a reduce radial profile. In examples, the implant device 1002 can be disposed within the shaft/sheath 1012(A) in an at least partially untwisted/straight (preloaded) or twisted state. In the figures, the device 1002 is shown in an untwisted state. In the compressed delivery configuration, the device 1002 can be somewhat more elongated compared to a fully expanded configuration thereof due to at least some of the struts/cells of the anchoring feature 1002(A)/1002(B) being deflected into more longitudinally-oriented configurations when radially crimped/compressed.

In some cases, the delivery system 1012 can comprise a pusher shaft 1012(C), which can be slidingly disposed within the outer sheath 1012(A) proximal and/or adjacent to the implant device 1002. The pusher 1012(C) can be configured to be used to push/advance the implant device 1002 relative to the outer shaft/sheath 1012(A) as a means to deploy the device 1002 from the sheath 1012(A). For example, the pusher 1012(C) can be distally advanced relative to the outer sheath 1012(A) to cause distal advancement of the compliance-enhancement implant device 1002 through a distal opening in the outer sheath/shaft 1012(A). Alternatively, or additionally, the implant device 1002 can be deployed from the outer sheath 1012(A) at least in part by proximally pulling the outer sheath 1012(A) relative to the pusher 1012(C).

In examples, delivery system 1012 can include one or more coupling/attachment elements to releasably attach to the implant device 1002. For example, the pusher 1012(C) or other component(s) of the delivery system 1012 can comprise one or more feet or arms that project distally and/or radially from the pusher 1012(C). In some cases, the pusher 1012(C) is releasably attached to the implant device 1002, wherein after the device 1002 has been deployed from the sheath 1012(A), positioned in the desired implantation site/position, and/or expanded, the pusher 1012(C) (or other component of the delivery system 1012) can be disengaged from the implant device 1002 to release the device 1002 and allow for removal/withdrawal of the delivery system 1012.

At block 906, the process 900 involves advancing the delivery system 1012 over the guidewire 1004 until the target implantation site is reached to thereby position the implant device 1002 for deployment in the target anatomy. In this example, the delivery system 1012 is advanced to a position within the descending aorta 1006; however, other target sites can be used.

At block 908, the process 900 involves deploying the implant device 1002 from the delivery system 1012. In examples, to deploy the implant device 1002, the outer sheath 1012(A) is proximally pulled and/or the pusher 1012(C) is distally pushed to thereby draw the sheath 1012(A) past the distal end of the implant device 1002, at least partially exposing/deploying the implant device 1002. As shown in image 1018a, initially the sheath 1012(A) can be withdrawn to position the first end/anchoring feature 1002(A) at a first position/location within the aorta 1006 (e.g., attach the first anchoring feature 1002(A) to a first internal portion of the aorta 1006), while maintaining position around and holding the second end/anchoring feature 1002(B). The sheath 1012(A) can be further withdrawn to position the second end/anchoring feature 1002(B) at a second/proximal position within the aorta 1006 (e.g., attach the second anchoring feature 1002(B) to a second internal portion of the aorta 1006), as shown in image 1018b. The implant device 1002 can comprise one or more radiopaque markers that can be referenced to determine/confirm the position of the implant device 1002 at various stage(s) of the process 900 using a suitable imaging modality.

In examples, the implant device 1002 is deployed when the device 1002 is untwisted (e.g., rotated away from a biased position). For instance, where the implant device 1002 is biased towards a twisted state, the device 1002 can be disposed in the delivery system 1012 in an at least partially untwisted state such that the device 1002 exerts a torsional force, as shown in the image 1014 in FIG. 10-1 where the torsion elements 1002(C) are in a substantially straight. As the first anchoring feature 1002(A) and/or the second anchoring feature 1002(B) are released from the delivery system 1012 and/or attach to the aorta 1006 (as shown in the images 1018a and 1018b), the torsional force of the device 1002 can be released to cause the implant device 1002 and/or the aorta 1006 to twist (as shown in image 1020, discussed in further detail below). This can set the device 1002 to a biased state/position. Thus, the implant device 1002 can be implanted on the aorta 1006 to bias the aorta 1006 to a twisted state (e.g., spring-load the aorta 1006).

Further, in examples, the aorta 1006 can be manipulated to position the aorta 1006 in the desired orientation for deployment of the implant device 1002. For instance, where the implant device 1002 is biased towards a twisted state, the device 1002 can be positioned in the delivery system 1012, held by a physician, or otherwise provided in the biased/twisted state (e.g., with little to no torsional force being applied). The implant device 1002 can be implanted on the aorta 1006 while a physician or device (e.g., the delivery system 1012 or another system) twists the aorta 1006. Thus, the implant device 1002 can be deployed while the aorta 1006 is twisted.

As noted herein, the anchoring features 1002(A) and/or 1002(B) can be implemented in a variety of manners. For instance, the anchoring features 1002(A) and/or 1002(B) can be self-expandable, such that the anchoring features 1002(A) and/or 1002(B) are expanded/fully deployed (e.g., anchored/secured to the internal tissue of the aorta 1006) upon release from the sheath 1012(A). For instance, expansion of the anchoring features 1002(A) and/or 1002(B) can be achieved via shape memory features of the anchoring features 1002(A) and/or 1002(B) and/or other portions of the device 1002. To illustrate, one or more portions of the device 1002 can comprise nitinol or other shape-memory metal configured to self-expand when released from the delivery sheath/capsule.

Alternatively, or additionally, the anchoring features 1002(A) and/or 1002(B) can be balloon/dilator expandable, such that a balloon/dilator are used to radially expand the anchoring features 1002(A) and/or 1002(B). The balloon/dilator can be implemented as part of or separately from the delivery system 1012. The balloon/dilator can be inserted through the anchoring features 1002(A) and/or 1002(B) to radially expand the anchoring features 1002(A) and/or 1002(B).

Alternatively, or additionally, the anchoring features 1002(A) and/or 1002(B) can be expanded/dilated using pull wire(s) that are configured to be pulled or pushed to cause expansion of the anchoring features 1002(A) and/or 1002(B). For example, a pull wire(s) can be coupled to the distal portion of the anchoring features 1002(A)/1002(B) such that pulling the wire(s) proximally causes the ends of the anchoring features 1002(A)/1002(B) to be brought together, thereby dilating/expanding the anchoring features 1002(A)/1002(B).

At block 910, the process 900 involves withdrawing the delivery system 1012 and/or guidewire 1004, leaving the implant device 1002 implanted in the aorta 1006, as shown in image 1020. With the implant device 1002 implanted, the increased compliance provided by the implant device 1002 can improve arterial blood flow and/or prevent elevated blood pressure. Other benefits can also be achieved, as described herein. Once fully deployed, the luminal pressure within the aorta 1006 can control a state of the implant device 1002. That is, the implant device 1002 can twist and untwist based on the luminal/aortic pressure in the aorta 1006.

Although many instances of the example process 900 are discussed in the context of deploying the implant device 1002 within the aorta 1006, the implant device 1002 can be implanted around the aorta 1006. For example, the delivery system 1012 or another device/system can be controlled to puncture one or more holes from the inside of the aorta 1006 to the outer surface. One or more portions of the anchoring features 1002(A) and/or 1002(B) can pass through the one or more holes to attach around the aorta 1006. As such, the implant device 1002 can be positioned such that some portions are within the aorta 1006 and other portions are outside of the internal blood path. Further, in some cases, an internal anchoring feature is attached to an external anchoring feature to facilitate a secure connection to the aorta 1006. Moreover, in some instances, one or more portions of the implant device 1002 can be secured to an outer surface/portion of the aorta 1006 and/or an inner surface/portion of the aorta 1006 in a procedure where the aorta 1006 is accessed through an incision near the target implantation site.

FIG. 11 illustrates an example implant device 1102 (which can be similar to or the same as any of the implant devices discussed herein) in a fully deployed state within the aorta 1104 (also referred to as internal anchoring). Here, anchoring features 1102(A) and 1102(B) are implemented as stents/wireframes that are expanded to anchor/secure the anchoring features 1102(A) and 1102(B) to internal walls/tissue of the aorta 1104. In other examples, the anchoring features 1102(A) and/or 1102(B) can be implemented with other types of features and/or the device 1102 can be anchored to other anatomical features/tissue.

FIG. 12 illustrates an example implant device 1202 (which can be similar to or the same as any of the implant devices discussed herein) in a fully deployed state around the aorta 1204 (also referred to outer/external anchoring). Here, anchoring features 1202(A) and 1202(B) are implemented as bands that are configured to wrap around the aorta 1204 to anchor/secure the anchoring features 1202(A) and 1202(B) to outer/external walls/tissue/portion of the aorta 1204. In other examples, the anchoring features 1202(A) and/or 1202(B) can be implemented with other types of features and/or the device 1202 can be anchored to other anatomical features/tissue.

In examples, one or more of the devices/systems discussed herein can be sterilized using one or more sterilization processes.

### ADDITIONAL EXAMPLES

1. An implant device including: one or more anchoring structures configured to anchor the implant device to a fluid vessel; and a torsion element coupled to the one or more anchoring structures and configured to cause the implant device to apply a torsional force to at least a first anchoring structure of the one or more anchoring structures when the first anchoring structure is rotated away from a biased position.

2. The implant device of any example herein, in particular example 1, wherein the torsion element includes a spring configured to bias the first anchoring structure to the biased position, the biased position being a first rotational position relative to a second anchoring structure of the one or more anchoring structures.

3. The implant device of any example herein, in particular examples 1 to 2, wherein the fluid vessel is an aorta.

4. The implant device of any example herein, in particular examples 1 to 3, wherein the implant device is configured to bias the fluid vessel to a twisted state.

5. The implant device of any example herein, in particular examples 1 to 4, wherein the implant device is configured to cause the fluid vessel to twist as a luminal pressure within the fluid vessel decreases and allow the fluid vessel to straighten as the luminal pressure increases.

6. The implant device of any example herein, in particular examples 1 to 5, wherein an amount of rotation of the first anchoring structure relative to the biased position is based at least in part on an amount of luminal pressure within the fluid vessel.

7. The implant device of any example herein, in particular examples 1 to 6, wherein the implant device is configured to cause the fluid vessel to twist during a first cardiac phase and untwist during a second cardiac phase.

8. The implant device of any example herein, in particular examples 1 to 7, wherein the one or more anchoring structures include a first anchoring structure and a second anchoring structure, the torsion element being coupled between the first anchoring structure and the second anchoring structure.

9. The implant device of any example herein, in particular examples 1 to 8, wherein the one or more anchoring structures include one or more stents.

10. The implant device of any example herein, in particular examples 1 to 9, wherein the one or more anchoring structures include one or more bands.

11. The implant device of any example herein, in particular examples 1 to 11, wherein the one or more anchoring structures are configured to anchor to internal tissue of the fluid vessel.

12. The implant device of any example herein, in particular examples 1 to 11, wherein the one or more anchoring structures are configured to anchor around an outer surface of the fluid vessel.

13. The implant device of any example herein, in particular examples 1 to 12, further including: a cylindrical tube coupled to or integral with the one or more anchoring structures and configured to twist.

14. The implant device of any example herein, in particular examples 1 to 13, wherein the implant device is configured to implement a compressed state for transcatheter delivery.

15. The implant device of any example herein, in particular examples 1 to 14, wherein the implant device is sterilized.

16. A device including: a first anchor configured to couple to a first location on a fluid vessel; a second anchor configured to couple to a second location on the fluid vessel; and a spring coupled between the first anchor and the second anchor and configured to cause the fluid vessel to twist during a first cardiac phase and untwist during a second cardiac phase.

17. The device of any example herein, in particular example 16, wherein the fluid vessel is an aorta.

18. The device of any example herein, in particular examples 16 to 17, wherein the first cardiac phase is diastole and the second cardiac phase is systole.

19. The device of any example herein, in particular examples 16 to 18, wherein the spring is configured to bias the fluid vessel to a twisted state.

20. The device of any example herein, in particular examples 16 to 19, wherein the first anchor includes a stent.

21. The device of any example herein, in particular examples 16 to 20, wherein the first anchor includes a band.

22. The device of any example herein, in particular examples 16 to 21, wherein the first anchor is configured to anchor to an internal portion of the fluid vessel.

23. The device of any example herein, in particular examples 16 to 22, wherein the first anchor is configured to anchor around an outer portion of the fluid vessel.

24. The device of any example herein, in particular examples 16 to 23, further including: an elastic tube coupled to or integral with at least one of the first anchor or the second anchor, the elastic tube being configured to twist.

25. The device of any example herein, in particular examples 16 to 24, wherein the device is configured to implement a compressed state for transcatheter delivery.

26. The device of any example herein, in particular examples 16 to 25, wherein the device is sterilized.

27. A method including: advancing an implant device through a fluid vessel, the implant device including a first anchoring element, a second anchoring element, and a torsion element coupled between the first anchoring element and the second anchoring element; and deploying the first anchoring element and the second anchoring element within the fluid vessel, the torsion element being configured to bias the fluid vessel to a twisted state.

28. The method of any example herein, in particular example 27, wherein the deploying includes anchoring at least one of the first anchoring element or the second anchoring element to an internal surface within the fluid vessel.

29. The method of any example herein, in particular examples 27 to 28, wherein the deploying includes anchoring at least one of the first anchoring element or the second anchoring element to an outer surface of the fluid vessel.

30. The method of any example herein, in particular examples 27 to 29, wherein the torsion element includes a spring configured to bias the fluid vessel to the twisted state.

31. The method of any example herein, in particular examples 27 to 30, wherein the fluid vessel is an aorta.

32. The method of any example herein, in particular examples 27 to 31, wherein the implant device is configured to cause the fluid vessel to straighten as luminal pressure increases within the fluid vessel.

33. The method of any example herein, in particular examples 27 to 32, wherein an amount of rotation of the first anchoring element relative to the second anchoring element is based at least in part on an amount of luminal pressure within the fluid vessel.

34. The method of any example herein, in particular examples 27 to 33, wherein the implant device is configured to bias the fluid vessel to the twisted state during a diastolic phase of a cardiac cycle.

35. The method of any example herein, in particular examples 27 to 34, wherein the first anchoring element includes a stent.

36. The method of any example herein, in particular examples 27 to 35, wherein the first anchoring element includes a band.

37. The method of any example herein, in particular examples 27 to 36, wherein the implant device includes an elastic tube coupled to or integral with at least one of the first anchoring element or the second anchoring element.

38. The method of any example herein, in particular examples 27 to 37, wherein the advancing includes advancing the implant device in a compressed state.

39. The method of any example herein, in particular examples 27 to 38, wherein the implant device is sterilized.

Depending on the example, certain acts, events, or functions of any of the processes or algorithms described herein can be performed in a different sequence, can be added, merged, and/or left out altogether. Thus, in certain examples, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain examples include, while other examples do not include, certain features, elements, and/or steps. Thus, such conditional language is not generally intended to imply that features, elements, and/or steps are in any way required for one or more examples or that one or more examples necessarily include logic for deciding, with or without author input or prompting, whether these features, elements, and/or steps are included or are to be performed in any particular example. The terms “comprising,” “including,” “having,” and the like are generally synonymous, used in their ordinary sense, and used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y, and Z,” unless specifically stated otherwise, is understood with the context as used in general to convey that an item, term, element, etc. can be either X, Y, or Z. Thus, such conjunctive language is not generally intended to imply that certain examples require at least one of X, at least one of Y, and at least one of Z to each be present.

In examples, various features are sometimes grouped together in a single example, figure, or description thereof for the purpose of streamlining the disclosure and aiding in the understanding of one or more of the various aspects. This method of disclosure, however, is not to be interpreted as reflecting an intention that any claim require more features than are expressly recited in that claim. Moreover, any components, features, or steps illustrated and/or described in a particular example herein can be applied to or used with any other example(s). Further, no component, feature, step, or group of components, features, or steps are necessary or indispensable for each example. Thus, it is intended that the scope of the subject matter herein disclosed and claimed below should not be limited by the particular examples described herein.

Certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather can generally distinguish the element from another element having a similar or identical name (but for use of the ordinal term). In addition, as used herein, indefinite articles (“a” and “an”) can indicate “one or more” rather than “one.” Further, an operation performed “based on” a condition or event can also be performed based on one or more other conditions or events not explicitly recited.

Unless otherwise defined, terms (including technical and/or scientific terms) used herein can have the same meaning as commonly understood by one of ordinary skill in the art to which examples belong. Terms, such as those defined in commonly used dictionaries, can be interpreted as having a meaning that is consistent with their meaning in the context of the relevant art and not be interpreted in an idealized or overly formal sense unless expressly so defined herein.

The spatially relative terms “outer,” “inner,” “upper,” “lower,” “below,” “above,” “vertical,” “horizontal,” and similar terms, can be used herein for ease of description to describe the relations between one element or component and another element or component as illustrated in the drawings. Spatially relative terms can encompass different orientations of the device in use or operation, in addition to the orientation depicted in the drawings. For example, in the case where a device shown in the drawing is turned over, the device positioned “below” or “beneath” another device can be placed “above” another device. Accordingly, the illustrative term “below” can include both the lower and upper positions. The device can also be oriented in the other direction, and thus the spatially relative terms can be interpreted differently depending on the orientations.

Unless otherwise expressly stated, comparative and/or quantitative terms, such as “less,” “more,” “greater,” and the like, can encompass the concepts of equality. For example, “less” can mean not only “less” in the strictest mathematical sense, but also “less than or equal to.”

What is claimed is:

1. A method of improving blood flow through an aorta, including:
advancing an implant device through an aorta, the implant device including a first anchoring element, a second anchoring element, and a torsion element coupled between the first anchoring element and the second anchoring element, the first and second anchoring elements each having a stent structure; and
deploying the first anchoring element and the second anchoring element within the aorta;
wherein the torsion element causes the first anchoring element to rotate relative to the second anchoring element for biasing the aorta toward a twisted state.

2. The method of claim 1, wherein the deploying includes anchoring at least one of the first anchoring element or the second anchoring element to an internal surface within the aorta.

3. The method of claim 1, wherein the deploying includes anchoring at least one of the first anchoring element or the second anchoring element to an outer surface of the aorta.

4. The method of claim 1, further comprising:
disposing the implant device in a delivery system in an at least partially untwisted state;
wherein the advancing includes advancing the delivery system through the aorta.

5. The method of claim 1, wherein the torsion element includes a spring to bias the aorta to the twisted state.

6. The method of claim 1, wherein the implant device causes the aorta to untwist as luminal pressure increases within the aorta.

7. A method including:
providing a delivery system;
disposing an implant device within the delivery system, the implant device including a first anchoring element, a second anchoring element, and a torsion element coupled between the first anchoring element and the second anchoring element;
advancing the delivery system to a target site of an aorta; and
using the delivery system to deploy the first anchoring element and the second anchoring element at the target site such that the implant device biases the aorta to a twisted state.

8. The method of claim 7, wherein the disposing includes disposing the implant device in the delivery system in an at least partially untwisted state.

9. The method of claim 7, wherein the implant device causes the aorta to twist during a diastolic phase of a cardiac cycle.

10. The method of claim 7, wherein the first anchoring element includes a stent.

11. The method of claim 7, wherein the first anchoring element includes a band.

12. The method of claim 7, wherein the implant device includes an elastic tube coupled to or integral with at least one of the first anchoring element or the second anchoring element.

13. The method of claim 7, wherein the disposing includes disposing the implant device within the delivery system in a compressed state.

14. A method including:
advancing an implant device to a target site of an aorta, the implant device including a first anchoring structure and a torsion element to apply a torsional force to the first anchoring structure when the first anchoring structure is rotated away from a biased state; and
deploying the first anchoring structure at the target site to cause the implant device to twist the aorta during a diastolic phase of a cardiac cycle.

15. The method of claim 14, wherein the deploying includes anchoring the first anchoring structure to an internal surface of the aorta.

16. The method of claim 14, wherein the deploying includes anchoring the first anchoring structure to an outer surface of the aorta.

17. The method of claim 14, wherein the torsion element includes a spring to bias the aorta to a twisted state.

18. The method of claim 14, wherein the implant device causes the aorta to untwist as luminal pressure increases within the aorta.

19. The method of claim 14, wherein the implant device includes a second anchoring structure, and the torsion element includes a straight bar attached to a first location on the first anchoring structure and a second location on the second anchoring structure, the first location is rotationally offset relative to the second location when the implant device is in the biased state.

20. The method of claim 14, wherein the implant device is sterilized.
